(19) United States
(12) Patent Application Publication
(10) Pub. No.: US 2017/0156629 A1
(43) Pub. Date: Jun. 8, 2017

(54) MEDICAL IMAGING APPARATUS AND METHOD OF PROCESSING MEDICAL IMAGE

(71) Applicant: Samsung Electronics Co., Ltd, Gyeonggi-do (KR)

(72) Inventors: Hyun-hee Jo, Gyeonggi-do (KR); Se-jin Yoo, Seoul (KR)

(21) Appl. No.: 15/344,319

(22) Filed: Nov. 4, 2016

(30) Foreign Application Priority Data
Dec. 8, 2015 (KR) 10-2015-0174158

(51) Int. Cl. A61B 5/055; G01R 33/341; G01R 33/56; A61B 5/00; G01R 33/54

(52) U.S. Cl. CPC A61B 5/055; A61B 5/0555; A61B 5/704; A61B 5/708; A61B 5/742; A61B 5/7475; A61B 5/7225; G01R 33/546; G01R 33/5608; G01R 33/5601; G01R 33/341; A61B 2576/02

(57) ABSTRACT

A medical imaging apparatus and method capable of simultaneously displaying sagittal plane images and axial plane images of both breasts and navigating through the images . The medical imaging apparatus includes a data acquirer configured to acquire first volume data including first sections of a left breast and a right breast of an object, an image processor configured to generate second volume data including second sections of the left breast and the right breast of the object, based on the first volume data, generate a sagittal plane image and an axial plane image of the first breast and the second breast, based on the first volume data or the second volume data. A display is configured to display a user interface (UI) screen image including the sagittal plane image and the axial plane image.

### CROSS-REFERENCE TO RELATED APPLICATION(S) AND CLAIM OF PRIORITY

The present application is related to and claims the benefit of Korean Patent Application No. 10-2015-0174158, filed on Dec. 8, 2015, in the Korean Intellectual Property Office, the disclosure of which is incorporated herein in its entirety by reference.

### TECHNICAL FIELD

The present disclosure relates to medical image processing apparatuses and medical image processing methods performed by the medical image processing apparatuses. More particularly, the present disclosure relates to a medical image processing apparatus that provides a user interface (UI) enabling a user to simultaneously observe sections of both breasts, and a medical image processing method performed by the medical image processing apparatus.

### BACKGROUND

A medical imaging apparatus according to an embodiment of the present disclosure is an electronic apparatus capable of generating and processing various medical images. Medical imaging apparatuses are equipment for capturing images of an internal structure of an object. Medical imaging apparatuses capture and/or process images of the structural details of a human body, internal tissue thereof, and fluid flow within a human body, and show the captured and/or processed images to a user. A user such as a doctor may diagnose a health state or a disease of a subject by using a medical image output from a medical imaging apparatus. Medical imaging apparatuses display an image of breasts according to various methods so that a user may diagnose a disease generated in the breasts.

Examples of the medical imaging apparatuses may include a magnetic resonance imaging (MRI) apparatus. MRI apparatuses are apparatuses for photographing a subject by using a magnetic field, and are widely used to accurately diagnose a disease since the MRI apparatuses three-dimensionally show not only bones, but also discs, joints, nerves, and ligaments at a desired angle.

In this connection, in the case of a body part that is divided into a left side and a right side, such as breasts, a medical imaging apparatus capable of simultaneously displaying both breasts is required.

### SUMMARY

To address the above-discussed deficiencies, it is a primary object to provide a medical imaging apparatus capable of simultaneously displaying sagittal plane images and axial plane images of a plurality of parts of an object and navigating the images, and a method of operating the medical imaging apparatus.

Additional aspects will be set forth in part in the description which follows and, in part, will be apparent from the description, or may be learned by practice of the presented embodiments.

According to an aspect of the present disclosure, a medical imaging apparatus includes a data acquirer configured to acquire first volume data including first sections of a left breast and a right breast of an object; an image processor configured to generate second volume data including second sections of the left breast and the right breast of the object, based on the first volume data, generate a sagittal plane image of the left breast and the right breast, based on the first volume data or the second volume data, and generate an axial plane image including the left breast and the right breast, based on the first volume data or the second volume data; and a display configured to display a user interface (UI) screen image including the sagittal plane image and the axial plane image.

The display may be further configured to display at least one reference line on each of the sagittal plane image and the axial plane image.

The image processor may be further configured to generate the axial plane image corresponding to a location of the at least one reference line on the sagittal plane image.

The data processor may be further configured to generate a subtraction image corresponding to a difference between an axial plane image before injecting a contrast medium and an axial plane image after injecting a contrast medium. The subtraction image may be an image corresponding to a location of the at least one reference line on the sagittal plane image. The display may be further configured to display the subtraction image.

The sagittal plane image may include a first sagittal plane image corresponding to the left breast of the object and a second sagittal plane image corresponding to the right breast of the object. The image processor may be further configured to generate the first sagittal plane image corresponding to a location of a first reference line on the axial plane image, and to generate the second sagittal plane image corresponding to a location of a second reference line on the axial plane image.

The image processor is further configured to set a distance from a center line on the axial plane image to the first reference line to be identical to a distance from the center line on the axial plane image to the second reference line.

The image processor may be further configured to arrange a chest wall of the object on the first sagittal plane image and a chest wall of the object on the second sagittal plane image such that the two chest walls face each other.

The medical imaging apparatus may further include an input unit configured to receive a user input for designating a location of the at least one reference line. The data processor may be further configured to change the location of the at least one reference line based on the user input.

The input unit may be further configured to receive information about a location of a center line on the axial plane image.

When the first sections are sagittal sections, the second sections may be axial sections. When the first sections are axial sections, the second sections may be sagittal sections.

The image processor may be further configured to generate a plurality of sagittal plane images of the left breast and a plurality of sagittal plane images of the right breast, based on the first volume data or the second volume data, and to select, from among the plurality of sagittal plane images of the right breast, a sagittal plane image of the right breast that is most similar to a sagittal plane image of the left breast. The display may be further configured to display the selected sagittal plane image of the right breast and the sagittal plane image of the left breast.

According to an aspect of the present disclosure, a method of operating a medical imaging apparatus includes acquiring first volume data including first sections of a left breast and a right breast of an object; generating second volume data including second sections of the left breast and the right breast of the object, based on the first volume data; generating a sagittal plane image of the left breast and the right breast, based on the first volume data or the second volume data; generating an axial plane image of breasts including the left breast and the right breast, based on the first volume data or the second volume data; and displaying a user interface (UI) screen image including the sagittal plane image and the axial plane image.

The displaying of the UI screen image may include displaying at least one reference line on each of the sagittal plane image and the axial plane image.

The generating of the axial plane image may include generating the axial plane image corresponding to a location of the at least one reference line on the sagittal plane image.

The method may further include generating a subtraction image corresponding to a difference between an axial plane image before injecting a contrast medium and an axial plane image after injecting a contrast medium; and displaying the subtraction image. The subtraction image may be an image corresponding to a location of the at least one reference line on the sagittal plane image.

The sagittal plane image may include a first sagittal plane image corresponding to the left breast of the object and a second sagittal plane image corresponding to the right breast of the object. The generating of the sagittal plane image may include generating the first sagittal plane image corresponding to a location of a first reference line on the axial plane image; and generating the second sagittal plane image corresponding to a location of a second reference line on the axial plane image.

The method may further include setting a distance from a center line on the axial plane image to the first reference line to be identical to a distance from the center line on the axial plane image to the second reference line.

The method may further include arranging a chest wall of the object on the first sagittal plane image and a chest wall of the object on the second sagittal plane image such that the two chest walls face each other.

The method may further include receiving a user input for designating a location of the at least one reference line; and changing the location of the at least one reference line based on the user input.

The receiving of the user input may include receiving information about a location of a center line on the axial plane image.

When the first sections are sagittal sections, the second sections may be axial sections. When the first sections are axial sections, the second sections may be sagittal sections.

The generating of the sagittal plane image may include generating a plurality of sagittal plane images of the left breast and a plurality of sagittal plane images of the right breast, based on the first volume data or the second volume data. The displaying may include selecting, from among the plurality of sagittal plane images of the right breast, a sagittal plane image of the right breast that is most similar to a sagittal plane image of the left breast; and displaying the selected sagittal plane image of the right breast and the sagittal plane image of the left breast.

A non-transitory computer-readable recording medium may have recorded thereon a program for executing a method as described above.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a block diagram of a general magnetic resonance imaging (MRI) system;

FIG. 2 illustrates a block diagram of a communication unit included in the MRI system of FIG. 1;

FIG. 3 illustrates a block diagram of a medical imaging apparatus according to an embodiment of the present disclosure;

FIG. 4 illustrates a block diagram of a medical imaging apparatus according to an embodiment of the present disclosure;

FIG. 5 illustrates screen images that may be displayed on a display of a medical imaging apparatus according to an embodiment of the present disclosure;

FIGS. 6A and 6B illustrate screen images that may be displayed on a display of a medical imaging apparatus according to an embodiment of the present disclosure;

FIGS. 7A and 7B illustrate screen images that may be displayed on a display of a medical imaging apparatus according to an embodiment of the present disclosure;

FIG. 8 illustrates screen images that may be displayed on a display of a medical imaging apparatus according to an embodiment of the present disclosure;

FIGS. 9A and 9B illustrate a change in a sagittal plane image according to a movement of a reference line on an axial plane image according to an embodiment of the present disclosure;

FIGS. 10A and 10B illustrate a change in a sagittal plane image according to a movement of a reference line on an axial plane image according to an embodiment of the present disclosure;

FIGS. 11A and 11B illustrate a change in an axial plane image according to a movement of a reference line on a sagittal plane image according to an embodiment of the present disclosure;

FIGS. 12A and 12B illustrate a change in a subtraction image according to a movement of a reference line on a sagittal plane image according to an embodiment of the present disclosure;

FIG. 13 illustrates a flowchart of a method of operating a medical imaging apparatus according to an embodiment of the present disclosure;

FIGS. 14A and 14B illustrate screen images that may be displayed on a display of a medical imaging apparatus according to an embodiment of the present disclosure;

FIGS. 15A and 15B illustrate sagittal plane images according to an embodiment of the present disclosure; and

FIG. 16 illustrates screen images that may be displayed on a display of a medical imaging apparatus according to an embodiment of the present disclosure.

### DETAILED DESCRIPTION

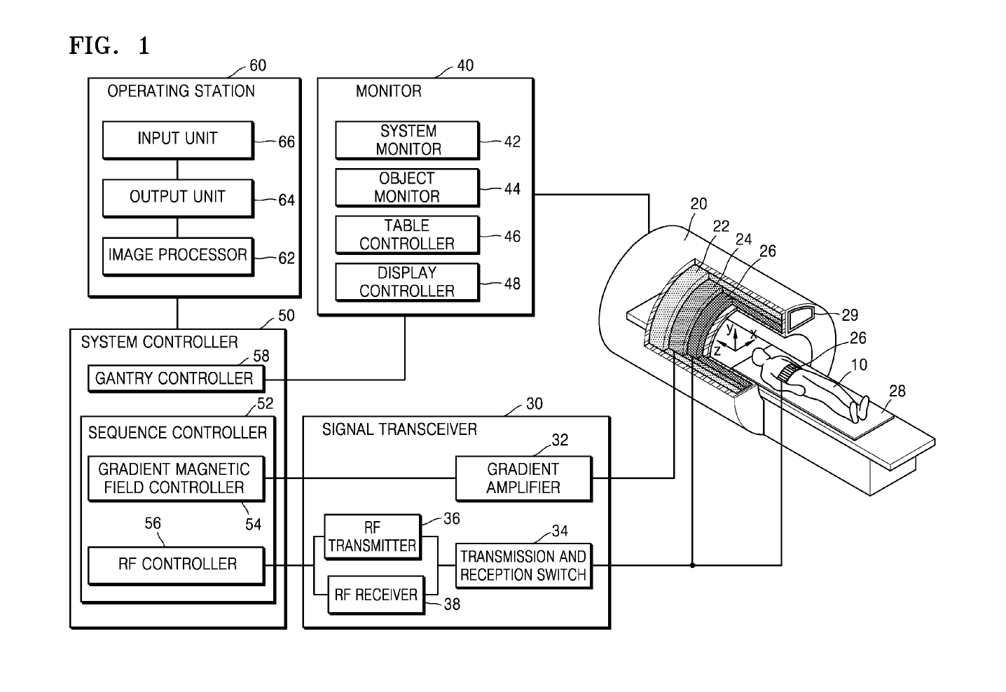

FIGS. 1 through 16, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

Advantages and features of one or more embodiments of the present disclosure and methods of accomplishing the same may be understood more readily by reference to the following detailed description of the embodiments and the accompanying drawings. In this regard, the present embodiments may have different forms and should not be construed as being limited to the descriptions set forth herein. Rather, these embodiments are provided so that this disclosure will be thorough and complete and will fully convey the concept of the present embodiments to one of ordinary skill in the art, and the present disclosure will only be defined by the appended claims.

Hereinafter, the terms used in the specification will be briefly described, and then the present disclosure will be described in detail.

The terms used in this specification are those general terms currently widely used in the art in consideration of functions regarding the present disclosure, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used herein have to be defined based on the meaning of the terms together with the description throughout the specification.

When a part “includes” or “comprises” an element, unless there is a particular description contrary thereto, the part can further include other elements, not excluding the other elements. Also, the term “unit” in the embodiments of the present disclosure means a software component or hardware component such as a field-programmable gate array (FPGA) or an application-specific integrated circuit (ASIC), and performs a specific function. However, the term “unit” is not limited to software or hardware. The “unit” may be formed so as to be in an addressable storage medium, or may be formed so as to operate one or more processors. Thus, for example, the term “unit” may refer to components such as software components, object-oriented software components, class components, and task components, and may include processes, functions, attributes, procedures, subroutines, segments of program code, drivers, firmware, micro codes, circuits, data, a database, data structures, tables, arrays, or variables. A function provided by the components and “units” may be associated with the smaller number of components and “units”, or may be divided into additional components and “units”.

Reference will now be made in detail to embodiments, examples of which are illustrated in the accompanying drawings, wherein like reference numerals refer to the like elements throughout. In the following description, well-known functions or constructions are not described in detail so as not to obscure the embodiments with unnecessary detail.

(Descriptions of Common Terms used in Specification)

In the present specification, an “image” may refer to multi-dimensional data composed of discrete image elements (e.g., pixels in a two-dimensional (2D) image and voxels in a three-dimensional (3D) image). For example, an image may be a medical image of an object acquired by an X-ray apparatus, a computed tomography (CT) apparatus, a magnetic resonance imaging (MRI) apparatus, an ultrasound diagnosis apparatus, or another medical imaging apparatus.

Furthermore, in the present specification, an “object” may be a human, an animal, or a part of a human or animal. For example, the object may be an organ (e.g., the liver, the heart, the womb, the brain, a breast, or the abdomen), a blood vessel, or a combination thereof. The object may be a phantom. The phantom means a material having a density, an effective atomic number, and a volume that are approximately the same as those of an organism. For example, the phantom may be a spherical phantom having properties similar to the human body.

Furthermore, in the present specification, a “user” may be, but is not limited to, a medical expert, such as a medical doctor, a nurse, a medical laboratory technologist, or a technician who repairs a medical apparatus.

Furthermore, in the present specification, an “MR image” refers to an image of an object obtained by using the nuclear magnetic resonance principle.

Furthermore, in the present specification, a “pulse sequence” refers to continuity of signals repeatedly applied by an MRI apparatus. The pulse sequence may include a time parameter of a radio frequency (RF) pulse, for example, repetition time (TR) or echo time (TE).

Furthermore, in the present specification, a “pulse sequence schematic diagram” shows an order of events that occur in an MRI apparatus. For example, the pulse sequence schematic diagram may be a diagram showing an RF pulse, a gradient magnetic field, an MR signal, or the like according to time.

(General Descriptions of Operations of MRI System)

An MRI system is an apparatus for acquiring a sectional image of a part of an object by expressing, in a contrast comparison, a strength of an MR signal with respect to a radio frequency (RF) signal generated in a magnetic field having a specific strength. For example, if an RF signal that only resonates a specific atomic nucleus (for example, a hydrogen atomic nucleus) is emitted for an instant toward the object placed in a strong magnetic field and then such emission stops, an MR signal is emitted from the specific atomic nucleus, and thus the MRI system may receive the MR signal and acquire an MR image. The MR signal denotes an RF signal emitted from the object. An intensity of the MR signal may be determined according to a density of a predetermined atom (for example, hydrogen) of the object, a relaxation time T1, a relaxation time T2, and a flow of blood or the like.

MRI systems include characteristics different from those of other imaging apparatuses. Unlike imaging apparatuses such as CT apparatuses that acquire images according to a direction of detection hardware, MRI systems may acquire 2D images or 3D volume images that are oriented toward an optional point. MRI systems do not expose objects or examiners to radiation, unlike CT apparatuses, X-ray apparatuses, position emission tomography (PET) apparatuses, and single photon emission CT (SPECT) apparatuses, may acquire images having high soft tissue contrast, and may acquire neurological images, intravascular images, musculoskeletal images, and oncologic images that are required to precisely capturing abnormal tissues.

(Descriptions of Elements of MRI System)

FIG. 1 illustrates a block diagram of a general MRI system. Referring to FIG. 1, the general MRI system may include a gantry 20, a signal transceiver 30, a monitoring unit 40, a system control unit 50, and an operating unit 60.

The gantry 20 prevents external emission of electromagnetic waves generated by a main magnet 22, a gradient coil 24, and an RF coil 26. A magnetostatic field and a gradient magnetic field are formed in a bore in the gantry 20, and an RF signal is emitted toward an object 10.

The main magnet 22, the gradient coil 24, and the RF coil 26 may be arranged in a predetermined direction of the gantry 20. The predetermined direction may be a coaxial cylinder direction. The object 10 may be disposed on a table 28 that is capable of being inserted into a cylinder along a horizontal axis of the cylinder.

The main magnet 22 generates a magnetostatic field or a static magnetic field for aligning magnetic dipole moments of atomic nuclei of the object 10 in a constant direction. A precise and accurate MR image of the object 10 may be obtained due to a magnetic field generated by the main magnet 22 being strong and uniform.

The gradient coil 24 includes X, Y, and Z coils for generating gradient magnetic fields in X-, Y-, and Z-axis directions crossing each other at right angles. The gradient coil 24 may provide location information of each region of the object 10 by differently inducing resonance frequencies according to the regions of the object 10.

The RF coil 26 may emit an RF signal toward a patient and receive an MR signal emitted from the patient. In detail, the RF coil 26 may transmit an RF signal having the same frequency as that of a precessional motion of atomic nuclei to the patient, stop transmitting the RF signal, and then receive an MR signal emitted from the patient.

For example, in order to transit an atomic nucleus from a low energy state to a high energy state, the RF coil 26 may generate and apply an electromagnetic wave signal that is an RF signal corresponding to a type of the atomic nucleus, to the object 10. When the electromagnetic wave signal generated by the RF coil 26 is applied to the atomic nucleus, the atomic nucleus may transit from the low energy state to the high energy state. Then, when electromagnetic waves generated by the RF coil 26 disappear, the atomic nucleus to which the electromagnetic waves were applied transits from the high energy state to the low energy state, thereby emitting electromagnetic waves having a Lamor frequency. In other words, when the applying of the electromagnetic wave signal to the atomic nucleus is stopped, an energy level of the atomic nucleus is changed from a high energy level to a low energy level, and thus the atomic nucleus may emit electromagnetic waves having a Lamor frequency. The RF coil 26 may receive electromagnetic wave signals from atomic nuclei included in the object 10.

The RF coil 26 may be realized as one RF transmitting and receiving coil having both a function of generating electromagnetic waves each having an RF that corresponds to a type of an atomic nucleus and a function of receiving electromagnetic waves emitted from an atomic nucleus. Alternatively, the RF coil 26 may be realized as a transmission RF coil having a function of generating electromagnetic waves each having an RF that corresponds to a type of an atomic nucleus, and a reception RF coil having a function of receiving electromagnetic waves emitted from an atomic nucleus.

The RF coil 26 may be fixed to the gantry 20 or may be detachable. When the RF coil 26 is detachable, the RF coil 26 may be an RF coil for a part of the object, such as a head RF coil, a chest RF coil, a leg RF coil, a neck RF coil, a shoulder RF coil, a wrist RF coil, or an ankle RF coil.

The RF coil 26 may communicate with an external apparatus via wires and/or wireles sly, and may also perform dual tune communication according to a communication frequency band.

The RF coil 26 may be a transmission exclusive coil, a reception exclusive coil, or a transmission and reception coil according to methods of transmitting and receiving an RF signal.

The RF coil 26 may be an RF coil having various numbers of channels, such as 16 channels, 32 channels, 72 channels, and 144 channels.

The gantry 20 may further include a display 29 disposed outside the gantry 20 and a display (not shown) disposed inside the gantry 20. The gantry 20 may provide predetermined information to the user or the object 10 through the display 29 and the display respectively disposed outside and inside the gantry 20.

The signal transceiver 30 may control the gradient magnetic field formed inside the gantry 20, i.e., in the bore, according to a predetermined MR sequence, and control transmission and reception of an RF signal and an MR signal.

The signal transceiver 30 may include a gradient amplifier 32, a transmission and reception switch 34, an RF transmitter 36, and an RF receiver 38.

The gradient amplifier 32 drives the gradient coil 24 included in the gantry 20, and may supply a pulse signal for generating a gradient magnetic field to the gradient coil 24 under the control of a gradient magnetic field controller 54. By controlling the pulse signal supplied from the gradient amplifier 32 to the gradient coil 24, gradient magnetic fields in X-, Y-, and Z-axis directions may be synthesized.

The RF transmitter 36 and the RF receiver 38 may drive the RF coil 26. The RF transmitter 36 may supply an RF pulse in a Lamor frequency to the RF coil 26, and the RF receiver 38 may receive an MR signal received by the RF coil 26.

The transmission and reception switch 34 may adjust transmitting and receiving directions of the RF signal and the MR signal. For example, the transmission and reception switch 34 may emit the RF signal toward the object 10 through the RF coil 26 during a transmission mode, and receive the MR signal from the object 10 through the RF coil 26 during a reception mode. The transmission and reception switch 34 may be controlled by a control signal output by an RF controller 56.

The monitoring unit 40 may monitor or control the gantry 20 or devices mounted on the gantry 20. The monitoring unit 40 may include a system monitoring unit 42, an object monitoring unit 44, a table controller 46, and a display controller 48.

The system monitoring unit 42 may monitor and control a state of the magnetostatic field, a state of the gradient magnetic field, a state of the RF signal, a state of the RF coil 26, a state of the table 28, a state of a device measuring body information of the object 10, a power supply state, a state of a thermal exchanger, and a state of a compressor.

The object monitoring unit 44 monitors a state of the object 10. In detail, the object monitoring unit 44 may include a camera for observing a movement or position of the object 10, a respiration measurer for measuring the respiration of the object 10, an electrocardiogram (ECG) measurer for measuring the electrical activity of the object 10, or a temperature measurer for measuring a temperature of the object 10.

The table controller 46 controls a movement of the table 28 where the object 10 is positioned. The table controller 46 may control the movement of the table 28 according to a sequence control of a sequence controller 52. For example, during moving imaging of the object 10, the table controller 46 may continuously or discontinuously move the table 28 according to the sequence control of the sequence controller 52, and thus the object 10 may be photographed in a field of view (FOV) larger than that of the gantry 20.

The display controller 48 controls the display 29 disposed outside the gantry 20 and the display disposed inside the gantry 20. In detail, the display controller 48 may control the display 29 and the display to be on or off, and may control a screen image to be output on the display 29 and the display. Also, when a speaker is located inside or outside the gantry 20, the display controller 48 may control the speaker to be on or off, or may control sound to be output via the speaker.

The system control unit 50 may include the sequence controller 52 for controlling a sequence of signals formed in the gantry 20, and a gantry controller 58 for controlling the gantry 20 and the devices mounted on the gantry 20.

The sequence controller 52 may include the gradient magnetic field controller 54 for controlling the gradient amplifier 32, and the RF controller 56 for controlling the RF transmitter 36, the RF receiver 38, and the transmission and reception switch 34. The sequence controller 52 may control the gradient amplifier 32, the RF transmitter 36, the RF receiver 38, and the transmission and reception switch 34 according to a pulse sequence received from the operating unit 60. Here, the pulse sequence includes all information required to control the gradient amplifier 32, the RF transmitter 36, the RF receiver 38, and the transmission and reception switch 34. For example, the pulse sequence may include information about a strength, an application time, and application timing of a pulse signal applied to the gradient coil 24.

The operating unit 60 may request the system control unit 50 to transmit pulse sequence information while controlling an overall operation of the MRI system.

The operating unit 60 may include an image processor 62 for receiving and processing the MR signal received by the RF receiver 38, an output unit 64, and an input unit 66.

The image processor 62 may process the MR signal received from the RF receiver 38 so as to generate MR image data of the object 10.

The image processor 62 receives the MR signal received by the RF receiver 38 and performs any one of various signal processes, such as amplification, frequency transformation, phase detection, low frequency amplification, and filtering, on the received MR signal.

The image processor 62 may arrange digital data in a k space (for example, also referred to as a Fourier space or a frequency space) of a memory, and rearrange the digital data into image data via 2D or 3D Fourier transformation.

The image processor 62 may perform a composition process or difference calculation process on image data if required. The composition process may include an addition process on a pixel or a maximum intensity projection (MIP) process. The image processor 62 may store not only the rearranged image data but also image data on which a composition process or a difference calculation process is performed, in a memory (not shown) or an external server.

The image processor 62 may perform any of the signal processes on the MR signal in parallel. For example, the image processor 62 may perform a signal process on a plurality of MR signals received by a multi-channel RF coil in parallel so as to rearrange the plurality of MR signals into image data.

The output unit 64 may output image data generated or rearranged by the image processor 62 to the user. The output unit 64 may also output information required for the user to manipulate the MRI system, such as a user interface (UI), user information, or object information. The output unit 64 may be a speaker, a printer, a cathode-ray tube (CRT) display, a liquid crystal display (LCD), a plasma display panel (PDP), an organic light-emitting device (OLED) display, a field emission display (FED), a light-emitting diode (LED) display, a vacuum fluorescent display (VFD), a digital light processing (DLP) display, a flat panel display (FPD), a 3-dimensional (3D) display, a transparent display, or any one of other various output devices that are well known to one of ordinary skill in the art.

The user may input object information, parameter information, a scan condition, a pulse sequence, or information about image composition or difference calculation by using the input unit 66. The input unit 66 may be a keyboard, a mouse, a track ball, a voice recognizer, a gesture recognizer, a touch screen, or any one of other various input devices that are well known to one of ordinary skill in the art.

The signal transceiver 30, the monitoring unit 40, the system control unit 50, and the operating unit 60 are separate components in FIG. 1, but it will be obvious to one of ordinary skill in the art that respective functions of the signal transceiver 30, the monitoring unit 40, the system control unit 50, and the operating unit 60 may be performed by another component. For example, the image processor 62 converts the MR signal received from the RF receiver 38 into a digital signal in FIG. 1, but alternatively, the conversion of the MR signal into the digital signal may be performed by the RF receiver 38 or the RF coil 26.

The gantry 20, the RF coil 26, the signal transceiver 30, the monitoring unit 40, the system control unit 50, and the operating unit 60 may be connected to each other by wire or wirelessly, and when they are connected wirelessly, the MRI system may further include an apparatus (not shown) for synchronizing clock signals therebetween. Communication between the gantry 20, the RF coil 26, the signal transceiver 30, the monitoring unit 40, the system control unit 50, and the operating unit 60 may be performed by using a high-speed digital interface, such as low voltage differential signaling (LVDS), asynchronous serial communication, such as a universal asynchronous receiver transmitter (UART), a low-delay network protocol, such as error synchronous serial communication or a controller area network (CAN), optical communication, or any of other various communication methods that are well known to one of ordinary skill in the art.

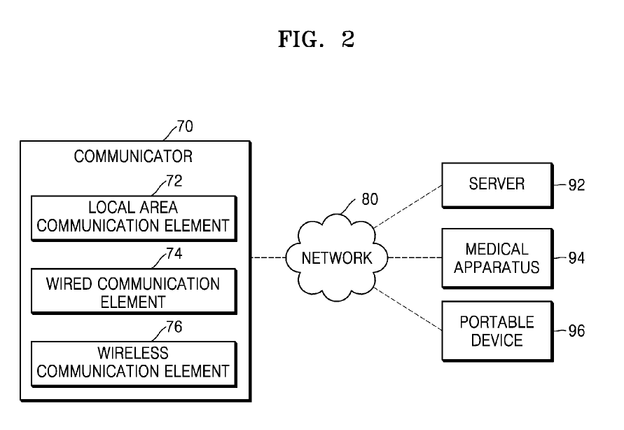

FIG. 2 illustrates a block diagram of a communication unit 70. Referring to FIG. 2, the communication unit 70 may be connected to at least one selected from the gantry 20, the signal transceiver 30, the monitoring unit 40, the system control unit 50, and the operating unit 60 of FIG. 1.

The communication unit 70 may transmit and receive data to and from a hospital server or another medical apparatus in a hospital, which is connected through a picture archiving and communication system (PACS), and perform data communication according to the digital imaging and communications in medicine (DICOM) standard.

As shown in FIG. 2, the communication unit 70 may be connected to a network 80 by wire or wirelessly to communicate with an external server 92, an external medical apparatus 94, or an external portable device 96.

In detail, the communication unit 70 may transmit and receive data related to the diagnosis of an object through the network 80, and may also transmit and receive a medical image captured by the external medical apparatus 94, such as a CT apparatus, an MRI apparatus, or an X-ray apparatus. In addition, the communication unit 70 may receive a diagnosis history or a treatment schedule of the object from the external server 92 and use the same to diagnose the object. The communication unit 70 may perform data communication not only with the external server 92 or the external medical apparatus 94 in a hospital, but also with the external portable device 96, such as a mobile phone, a personal digital assistant (PDA), or a laptop of a doctor or patient.

Also, the communication unit 70 may transmit information about a malfunction of the MRI system or about a medical image quality to a user through the network 80, and receive a feedback regarding the information from the user.

The communication unit 70 may include at least one component enabling communication with an external apparatus.

For example, the communication unit 70 may include a local area communication module 72, a wired communication module 74, and a wireless communication module 76. The local area communication module 72 refers to a module for performing local area communication with an apparatus within a predetermined distance. Examples of local area communication technology according to an embodiment of the present disclosure include, but are not limited to, a wireless local area network (LAN), WI-FI®, BLUETOOTH®, ZIGBEE®, Wi-Fi DIRECT® (WFD), ultra-wideband (UWB), infrared data association (IrDA), Bluetooth low energy (BLE), and near field communication (NFC).

The wired communication module 74 refers to a module for performing communication by using an electric signal or an optical signal. Examples of wired communication technology include wired communication techniques using a pair cable, a coaxial cable, and an optical fiber cable, and other well known wired communication techniques.

The wireless communication module 76 transmits and receives a wireless signal to and from at least one selected from a base station, an external apparatus, and a server in a mobile communication network. Here, the wireless signal may be a voice call signal, a video call signal, or data in any one of various formats according to transmission and reception of a text/multimedia message.

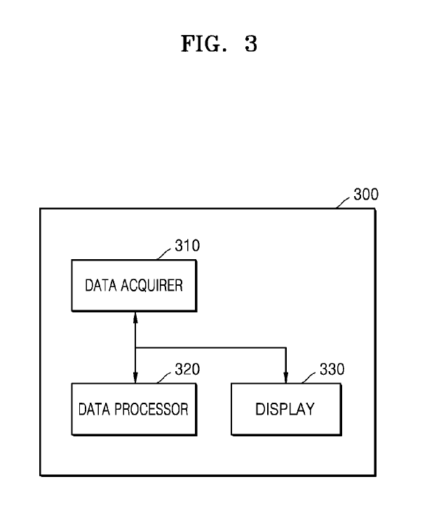

In this connection, a user needs to simultaneously check a plurality of parts of an object in some cases. For example, in the case of a body part that is divided into a left side and a right side, such as breasts, a user may need to simultaneously check two breasts in order to diagnose a bilateral breast lesion. Accordingly, a medical imaging apparatus capable of simultaneously displaying both right and left breasts is needed. A medical imaging apparatus capable of simultaneously displaying a sagittal plane image and an axial plane image of a plurality of parts of an object and navigating the images and a method of operating the medical imaging apparatus will now be described with reference to FIGS. 3-13.

FIG. 3 illustrates a block diagram of a medical imaging apparatus 300 according to an embodiment of the present disclosure.

The medical imaging apparatus 300 may be included in the MRI system described above with reference to FIG. 1. A data acquirer 310 may correspond to the signal transceiver 30 of FIG. 1. An data processor 320 may correspond to the image processor 62 of FIG. 1. A display 330 may correspond to the output unit 64 of FIG. 1. A repeated description thereof will be omitted. The data acquirer 310, the data processor 320, and the display 330 may be implemented as hardware.

The data acquirer 310 acquires first volume data including first sections of a first part and a second part of an object. The data acquirer 310 may correspond to the signal transceiver 30 of FIG. 1. The data acquirer 310 may acquire the first volume data by transmitting an RF signal to the object and receiving an RF signal emitted by the object.

The data acquirer 310 may acquire the first volume data from the communication unit 70 of FIG. 2. For example, the data acquirer 310 may acquire the first volume data from the external server 92, the external medical apparatus 94, and the external portable device 96 connected to the network 80.

The data acquirer 310 may acquire three-dimensional (3D) volume data from the object. For example, the data acquirer 310 may acquire the first volume data from the object in real time. The first volume data may be data previously acquired from the object. For example, the first volume data may be data stored in a storage unit (not shown), data acquired by the signal transceiver 30 of FIG. 1, or data received by the communication unit 70 of FIG. 2.

The first volume data may be comprised of a plurality of two-dimensional (2D) sections. For example, the first volume data may include a plurality of first sections. The first sections may be sagittal planes. The first sections may be axial planes. For example, the data acquirer 310 may acquire a plurality of sagittal sections, and the plurality of sagittal sections may be the first volume data.

The data acquirer 310 may acquire first volume data about a plurality of parts on the object. The plurality of parts may be the first part and the second part. The first part and the second part represent different locations on the object. For example, when the first part is a left breast, the second part may be a right breast.

The data processor 320 may generate second volume data including second sections of the first part and the second part of the object, based on the first volume data. The second sections may be sections that are not parallel to the first sections. For example, the second sections may be perpendicular to the first sections. When the first sections are sagittal sections, the second sections may be axial sections. When the first sections are axial sections, the second sections may be sagittal sections.

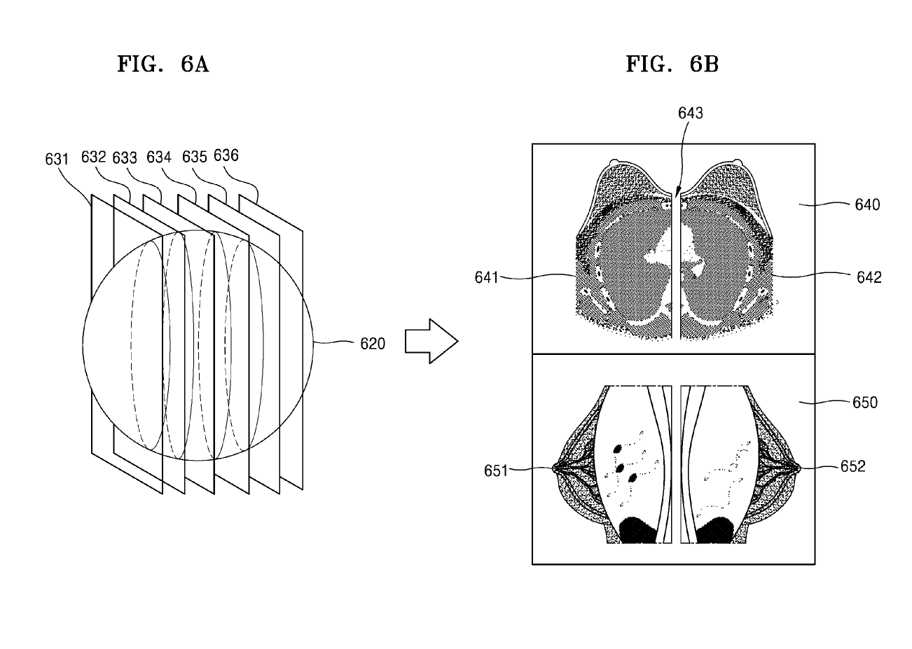

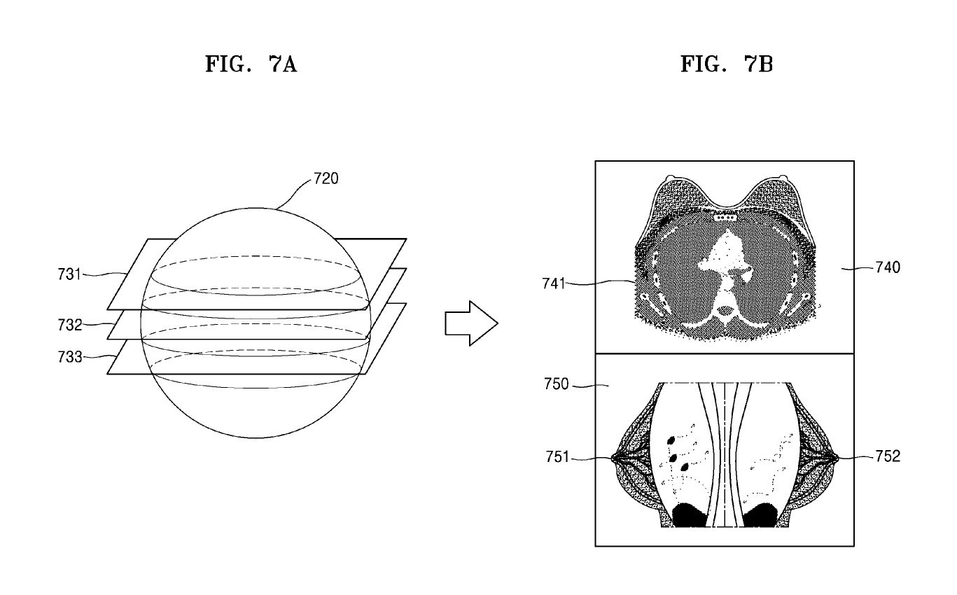

The second volume data may include the second sections. The first volume data and the second volume data may include data about the same part of the object. The first volume data and the second volume data may include different sections. This will be described in greater detail later with reference to FIGS. 6 and 7. Since the medical imaging apparatus 300 is able to transform the first volume data including the first section into the second volume data, the medical imaging apparatus 300 may show the object in a direction desired by a user.

The data processor 320 may generate a sagittal plane image of the first part and the second part of the object, based on the first volume data or the second volume data.

When the first sections included in the first volume data are sagittal sections, the data processor 320 may generate a sagittal plane image of the first part and the second part of the object, based on the first volume data.

When the second sections included in the second volume data are sagittal sections, the data processor 320 may generate the sagittal plane image of the first part and the second part of the object, based on the second volume data.

The data processor 320 may generate an axial plane image of a third part including the first part and the second part of the object, based on the second volume data or the first volume data.

When the first sections included in the first volume data are axial sections, the data processor 320 may generate the axial plane image of the third part of the object, based on the first volume data.

When the second sections included in the second volume data are axial sections, the data processor 320 may generate the axial plane image of the third part of the object, based on the second volume data.

The display 330 may display a user interface (UI) screen image including the sagittal plane image and the axial plane image.

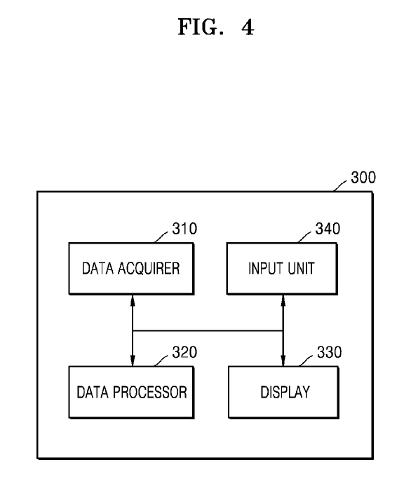

FIG. 4 illustrates a block diagram of a medical imaging apparatus 300 according to another embodiment of the present disclosure.

Referring to FIG. 4, the medical imaging apparatus 300 may include the data acquirer 310, the data processor 320, the display 330, and an input unit 340. Since the data acquirer 310, the data processor 320, and the display 330 have already been described above with reference to FIG. 3, a repeated description thereof is omitted herein.

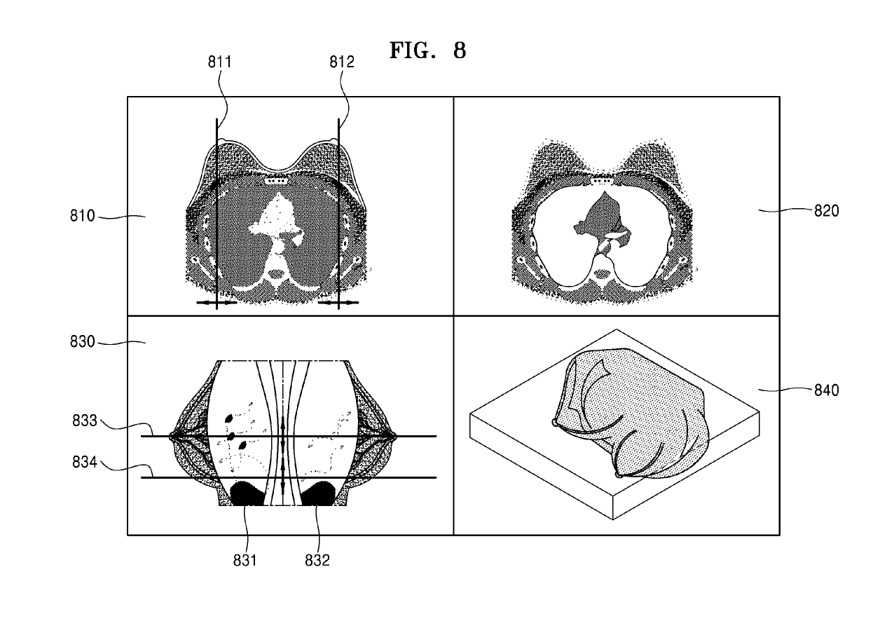

The input unit 340 may correspond to the input unit 66 of FIG. 1. The input unit 340 may receive a user input. The data processor 320 may change a location of a reference line, based on the user input. The reference line will be described in greater detail later with reference to FIGS. 8-12.

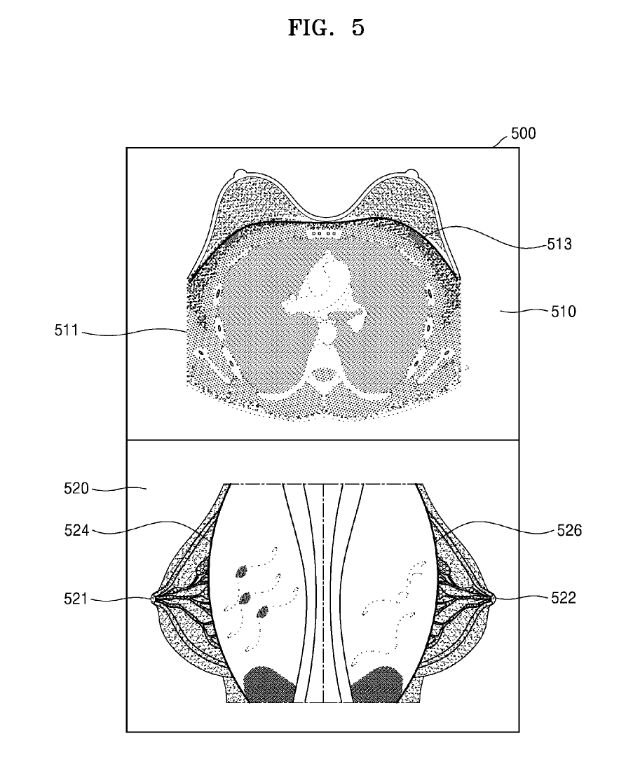

FIGS. 5-12 will now be described together with the medical imaging apparatus 300 described above with reference to FIG. 4.

FIG. 5 illustrates a screen image that may be displayed on the display 330 of the medical imaging apparatus 300 according to an embodiment of the present disclosure.

The display 330 may display a UI screen image 500. The UI screen image 500 may include a sagittal plane image 520 of an object and an axial plane image 510 thereof. The axial plane image 510 may include an axial section of an object 511. The axial plane image 510 may include an image including a first part and a second part of an object. For example, the axial plane image 510 may be an image including an image of a left breast of the object 511 and an image of a right breast thereof. The display 330 may also display a chest wall 513 together with the image of the left breast and the image of the right breast. The display 330 may indicate the chest wall 513 by using a bold line. The medical imaging apparatus 300 may determine a location of the chest wall 513. The medical imaging apparatus 300 may perform image processing to determine the location of the chest wall 513. For example, the medical imaging apparatus 300 may determine, as the chest wall 513, a line obtained by connecting points where pixel values suddenly significantly change. Since the axial plane image 510 may be divided into a breast area and a lung area by the chest wall 513, a user may easily observe the breast area.

The sagittal plane image 520 may include a sagittal section of the object 511. The sagittal plane image 520 may include an image 521 of the first part of the object and an image 522 of the second part thereof. For example, the first part may be a left breast of the object, and the second part may be a right breast thereof. The sagittal plane image 520 may include the image 521 of the left breast and the image 522 of the right breast. As shown in FIG. 5, the data processor 320 may arrange a chest wall of the object on a first sagittal plane image and a chest wall of the object on a second sagittal plane image such that the two chest walls face each other.

The display 330 may display a chest wall 524 on the image 521 of the left breast. The display 330 may also display a chest wall 526 on the image 522 of the right breast. The medical imaging apparatus 300 may perform image processing to determine the locations of the chest walls 524 and 526. The sagittal plane image 520 may be divided into a breast area and a lung area by the chest walls 524 and 526. The medical imaging apparatus 300 may adjust a distance between the chest wall 524 and the chest wall 526. The medical imaging apparatus 300 may downsize the lung area and display a downsized lung area on the display 330. A user may easily observe the breast area.

The display 330 may display the axial plane image 510 and the sagittal plane image 520 at the same time. The axial plane image 510 and the sagittal plane image 520 may be images acquired at the same time. A user may simultaneously see the images of the first part and the second part. The user may also simultaneously see images of different sections of the object. Accordingly, the user may easily diagnose a bilateral lesion.

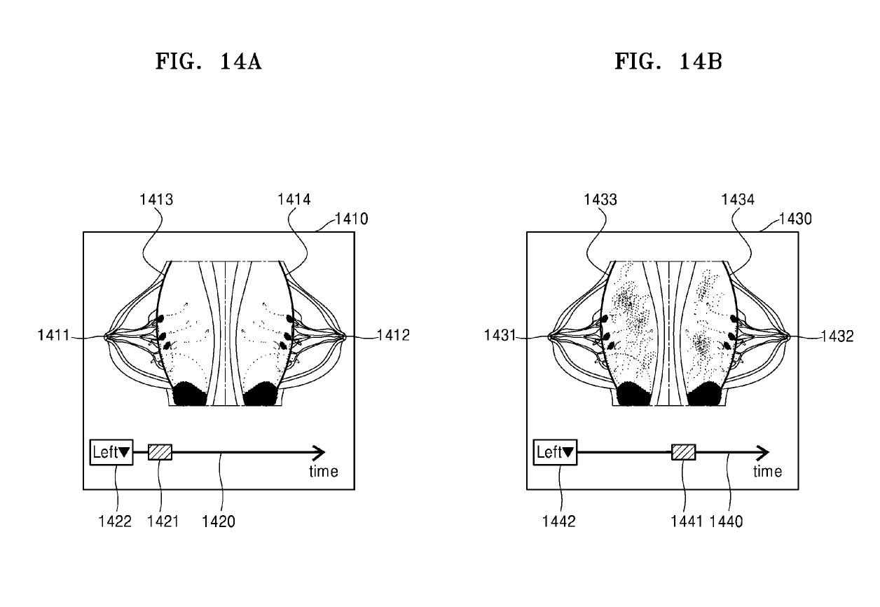

The display 330 may also display an image 521 of the left breast and an image 522 of the right breast which are acquired at different times. This will be described in greater detail with reference to FIG. 14.

FIGS. 6A and 6B explain screen images that may be displayed on the display 330 of the medical imaging apparatus 300 according to an embodiment of the present disclosure.

For convenience of explanation, FIG. 6A briefly expresses an object 620 as a sphere. In FIG. 6A, 6 sagittal sections 631, 632, 633, 634, 635, and 636 are displayed, but embodiments are not limited thereto. A number of sagittal sections smaller than 6 may be displayed or a number of sagittal sections greater than 6 may be displayed.

In FIG. 6A, the medical imaging apparatus 300 may acquire first volume data including first sections of the object 620. The first sections may be the sagittal sections 631, 632, 633, 634, 635, and 636. The first volume data may include data about a first part and a second part of the object 620.

The medical imaging apparatus 300 may generate second volume data, based on the first volume data. For example, the first volume data may include the sagittal sections 631, 632, 633, 634, 635, and 636. The medical imaging apparatus 300 may generate a plurality of axial sections, based on the plurality of sagittal sections 631, 632, 633, 634, 635, and 636. The medical imaging apparatus 300 may generate second volume data including the plurality of axial sections.

In FIG. 6B, the medical imaging apparatus 300 may display a sagittal plane image 650 on a UI screen image, based on the first volume data. The medical imaging apparatus 300 may also display an axial plane image 640 on the UI screen image, based on the second volume data.

The medical imaging apparatus 300 may display the sagittal plane image 650 of the first part and the second part of the object 620, on the UI screen image. For example, the sagittal plane image 650 may include a sagittal plane image 651 of the left breast and a sagittal plane image 652 of the right breast.

The sagittal plane image 650 of each part of FIG. 6B may correspond to the sagittal sections of FIG. 6A. For example, the sagittal plane image 651 of the left breast may correspond to the sagittal section 631 of FIG. 6A. The sagittal plane image 652 of the right breast may correspond to the sagittal section 634 of FIG. 6A.

The medical imaging apparatus 300 may display the axial plane image 640 of a third part including the first part and the second part of the object 620, on the UI screen image. The axial plane image 640 may include an axial plane image 641 of the left breast and an axial plane image 642 of the right breast. The axial plane image 640 may be based on the second volume data. The second volume data may be generated based on the first volume data. An artifact 643 may be generated during transformation of volume data. The artifact 643 may be removed via image processing.

The data processor 320 may generate sagittal plane images of a plurality of left breasts and sagittal plane images of a plurality of right breasts, based on the first volume data or the second volume data. The data processor 320 may select a sagittal plane image of a right breast that is the most similar to one of the sagittal plane images of the plurality of left breasts. The display 330 may display the selected sagittal plane image of the right breast and the sagittal plane image of the left breast that is the most similar to the selected sagittal plane image.

For example, the sagittal sections 631, 632, and 633 may correspond to the left breast. The sagittal sections 634, 635, and 636 may correspond to the right breast. The medical imaging apparatus 300 may generate a plurality of sagittal plane images of the left breast corresponding to the sagittal sections 631, 632, and 633, based on the first volume data. The medical imaging apparatus 300 may also generate a plurality of sagittal plane images of the right breast corresponding to the sagittal sections 634, 635, and 636, based on the first volume data. The data processor 320 may select a sagittal plane image of the right breast that is the most similar to the sagittal plane image of the left breast corresponding to the sagittal section 631. For example, the medical imaging apparatus 300 may select the sagittal plane image of the right breast corresponding to the sagittal section 635, based on similarity processing of images. The medical imaging apparatus 300 may determine that the sagittal plane image of the right breast corresponding to the sagittal section 635 is the most similar to that of the left breast corresponding to the sagittal section 631. The medical imaging apparatus 300 may also determine that the sagittal plane image of the right breast corresponding to the sagittal section 636 is the most similar to that of the left breast corresponding to the sagittal section 632.

The sagittal plane image of the left breast corresponding to the sagittal section 631 may correspond to the sagittal plane image 651 of the left breast of FIG. 6B. The sagittal plane image of the right breast corresponding to the sagittal section 635 may correspond to the sagittal plane image 652 of the right breast of FIG. 6B. The medical imaging apparatus 300 may display both the sagittal plane image 651 of the left breast and the sagittal plane image 652 of the right breast, which are similar to each other.

The medical imaging apparatus 300 may store a result of matching between a sagittal plane image of the left breast and a sagittal plane image of the right breast as described above. The medical imaging apparatus 300 may display the result of the matching on the display 330. For example, when a user selects the sagittal plane image 651 of the left breast corresponding to the sagittal section 631 via the input unit 340, the medical imaging apparatus 300 may automatically display the sagittal plane image 652 corresponding to the sagittal section 635.

As such, when the medical imaging apparatus 300 simultaneously displays a sagittal plane image of the left breast and a sagittal plane image of the right breast that is the most similar to the sagittal plane image of the left breast, a user may easily discover a lesion via a comparison between the left breast and the right breast.

FIGS. 7A and 7B explain screen images that may be displayed on the display 330 of the medical imaging apparatus 300 according to an embodiment of the present disclosure.

For convenience of explanation, FIG. 7A briefly expresses an object 720 as a sphere. In FIG. 7A, three axial sections 731, 732, and 733 are displayed, but embodiments are not limited thereto. A number of axial sections smaller than 3 may be displayed or a number of axial sections greater than 3 may be displayed.

In FIG. 7A, the medical imaging apparatus 300 may acquire first volume data including first sections of the object 720. The first sections may be the axial sections 731, 732, and 733. The first volume data may include data about a third part of the object 720. The third part may include a first part and a second part of the object 720.

The medical imaging apparatus 300 may generate second volume data, based on the first volume data. For example, the first volume data may include the axial sections 731, 732, and 733. The medical imaging apparatus 300 may generate a plurality of sagittal sections, based on the plurality of axial sections 731, 732, and 733. The medical imaging apparatus 300 may generate second volume data including the plurality of sagittal sections.

In FIG. 7B, the medical imaging apparatus 300 may display an axial plane image 740 on a UI screen image, based on the first volume data. The medical imaging apparatus 300 may also display a sagittal plane image 750 on the UI screen image, based on the second volume data.

The medical imaging apparatus 300 may display an axial plane image 740 of a third part including a first part and a second part of an object 741, on the UI screen image. The axial plane image 740 may be based on the first volume data.

The axial plane image 740 of each part of FIG. 7B may correspond to the axial sections of FIG. 7A. For example, the axial plane image 740 of the object 741 may correspond to the axial section 732 of FIG. 7A.

The medical imaging apparatus 300 may display a sagittal plane image 750 of the first part and the second part of the object 741, on the UI screen image. For example, the sagittal plane image 750 may include a sagittal plane image 751 of the left breast and a sagittal plane image 752 of the right breast.

The medical imaging apparatus 300 may display the sagittal plane image 750 on the UI screen image, based on the second volume data. The medical imaging apparatus 300 may generate the second volume data, based on the first volume data. The medical imaging apparatus 300 may generate one sagittal plane image 750 by arranging the sagittal plane image 751 of the left breast and the sagittal plane image 752 of the right breast such that chest walls of the sagittal plane image 751 and the sagittal plane image 752 face each other. A user may see both the axial plane image 740 and the sagittal plane image 750. The user may also see the sagittal plane image 751 of the left breast and the sagittal plane image 752 of the right breast at the same time. Accordingly, the user may easily diagnose a bilateral lesion.

FIG. 8 illustrates a screen image that may be displayed on the display 330 of the medical imaging apparatus 300 according to an embodiment of the present disclosure.

Referring to FIG. 8, the medical imaging apparatus 300 may display a UI screen image including an axial plane image 810, a subtraction image 820, a sagittal plane image 830, and a 3D image 840.

The display 330 of the medical imaging apparatus 300 may display at least one reference line on each of a sagittal plane image and an axial plane image. For example, the medical imaging apparatus 300 may display a first reference line 811 and a second reference line 812 on the axial plane image 810. The medical imaging apparatus 300 may also display a third reference line 833 and a fourth reference line 834 on the sagittal plane image 830.

The data processor 320 may generate an axial plane image 810 corresponding to a location of a reference line on the sagittal plane image 830. For example, the data processor 320 may generate an axial plane image 810 corresponding to a location of the third reference line 833 on the sagittal plane image 830. The display 330 may display the generated axial plane image 810. A user may change the location of the third reference line 833 via the input unit 340. When the location of the third reference line 833 is moved upward or downward, the medical imaging apparatus 300 may display a changed axial plane image 810 corresponding to the changed location of the third reference line 833.

The data processor 320 may generate a subtraction image 820 corresponding to a difference between an axial plane image before injecting a contrast medium and an axial plane image after injecting a contrast medium. The contrast medium is a substance that enhances the X-ray absorption contrast of tissues to enhance the visibility of tissues and/or blood vessels. Thus, when the axial plane image before injecting a contrast medium is subtracted from the axial plane image after injecting a contrast medium, the subtraction image 820 represents tissues and/or blood vessels more minutely.

The subtraction image 820 is an image corresponding to a location of a reference line on a sagittal plane image. For example, the data processor 320 may generate a subtraction image 820 corresponding to a location of the fourth reference line 834 on the sagittal plane image 830. The display 330 may display the generated subtraction image 820. A user may change the location of the fourth reference line 834 via the input unit 340. When the location of the fourth reference line 834 is moved upward or downward, the medical imaging apparatus 300 may display a changed subtraction image 820 corresponding to the changed location of the fourth reference line 834.

The sagittal plane image 830 may include a first sagittal plane image 831 corresponding to a first part of an object and a second sagittal plane image 832 corresponding to a second part of the object. The data processor 320 may generate a first sagittal plane image 831 corresponding to a location of the first reference line 811 on the axial plane image 810. The data processor 320 may also generate a second sagittal plane image 832 corresponding to a location of the second reference line 812 on the axial plane image 810. For example, the first sagittal plane image 831 may be a sagittal plane image of a left breast. The second sagittal plane image 832 may be a sagittal plane image of a right breast. The display 330 may display the first sagittal plane image 831 and the second sagittal plane image 832.

A user may correct the locations of the first reference line 811 and second reference line 812 via the input unit 340. The data processor 320 may generate a first sagittal plane image 831 corresponding to the changed location of the first reference line 811. The data processor 320 may also generate a second sagittal plane image 832 corresponding to the changed location of the second reference line 812. The data processor 320 may generate the sagittal plane image 830 including the first sagittal plane image 831 and the second sagittal plane image 832. The display 330 may display the sagittal plane image 830.

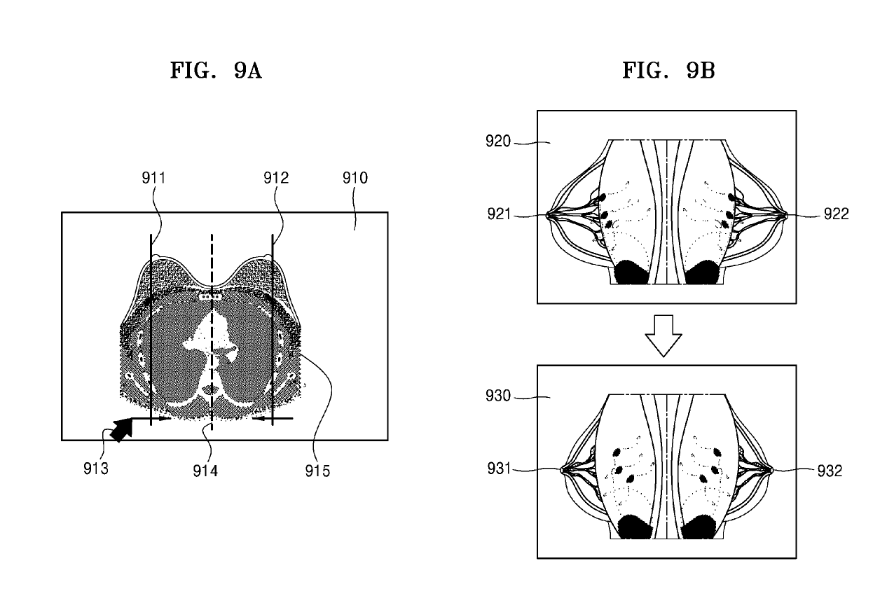

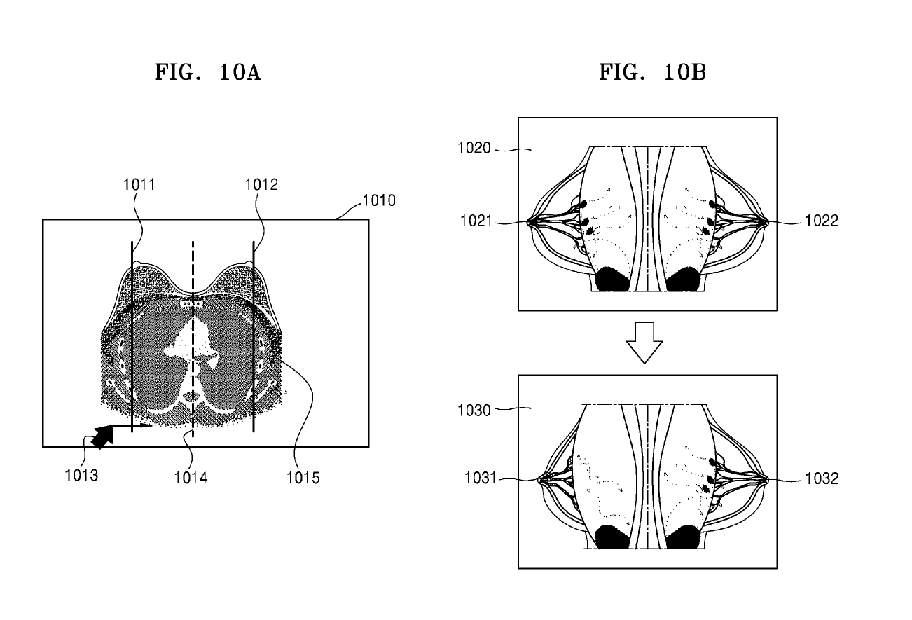

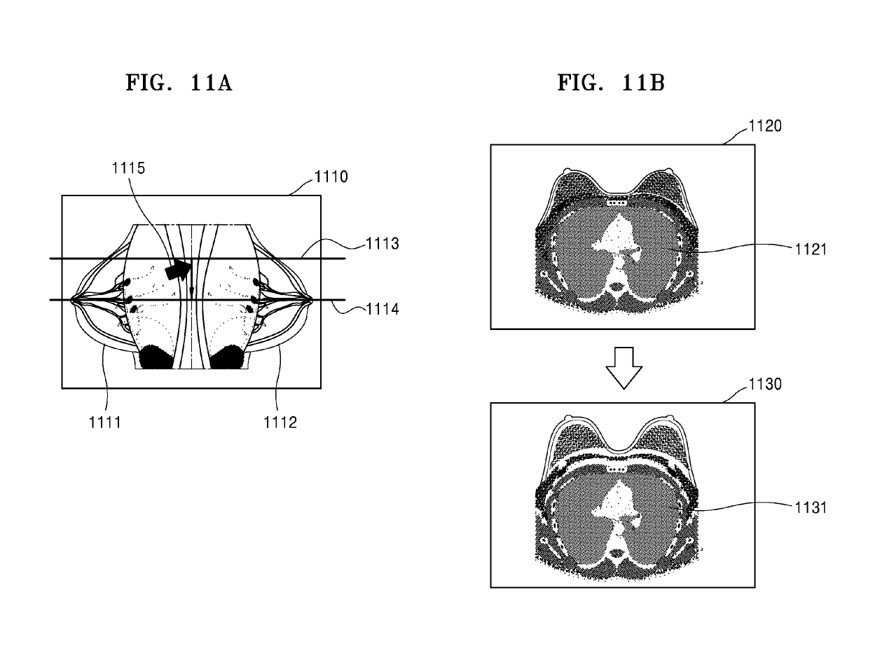

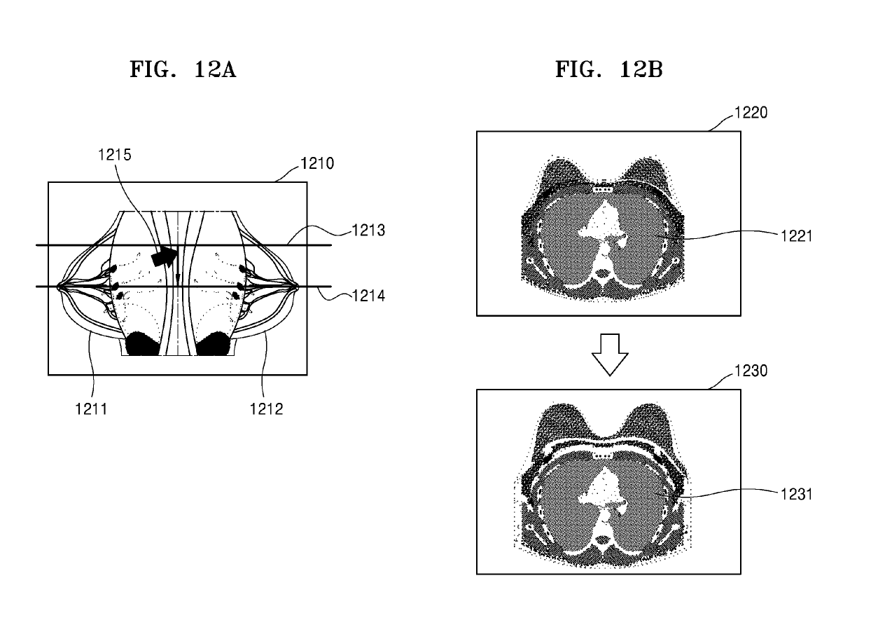

A change in a sagittal plane image according to a location of a reference line on an axial plane image will now be described in detail with reference to FIGS. 9 and 10. Changes in an axial plane image and a subtraction image according to a location of a reference line on a sagittal plane image will be described in detail with reference to FIGS. 11 and 12.

FIGS. 9A and 9B illustrate a change in a sagittal plane image according to a movement of a reference line on an axial plane image according to an embodiment of the present disclosure.

Referring to FIG. 9A, the medical imaging apparatus 300 may display an axial plane image 910 of an object 915. Although only the axial plane image 910 is displayed in FIG. 9A, the axial plane image 910 may be displayed together with a sagittal plane image, a subtraction image, and a 3D image as in FIG. 8.

The medical imaging apparatus 300 may display a center line 914 on the axial plane image 910. The medical imaging apparatus 300 may also display a first reference line 911 and a second reference line 912 on the axial plane image 910. The data processor 320 may set a distance from the center line 914 to the first reference line 911 to be identical to a distance from the center line 914 to the second reference line 912.

The input unit 340 may receive a user input. The data processor 320 may move an indicator 913, based on the user input. The input unit 340 may receive information about the location of the center line 914 on the axial plane image 910. The data processor 320 may change the location of the center line 914, based on the information about the location of the center line 914.

The input unit 340 may receive a user input for designating a location of a reference line on the axial plane image 910. The data processor 320 may change the location of the first reference line 911 and/or the location of the second reference line 912, based on the user input.

For example, when a user changes the location of the first reference line 911 by using the indicator 913, the data processor 320 may automatically change the location of the second reference line 912. Consequently, the data processor 320 may maintain the distance from the center line 914 to the first reference line 911 to be identical to the distance from the center line 914 to the second reference line 912.

Referring to FIG. 9B, the medical imaging apparatus 300 may display a sagittal plane image 920. Although only the sagittal plane image 920 is displayed in FIG. 9B, the sagittal plane image 920 may be displayed together with the axial plane image 910, a subtraction image, and a 3D image as in FIG. 8.

The data processor 320 may generate a first sagittal plane image 921 corresponding to the location of the first reference line 911 on the axial plane image 910. The data processor 320 may also generate a second sagittal plane image 922 corresponding to the location of the second reference line 912 on the axial plane image 910.

A user may change the location of the first reference line 911 by using the indicator 913. In this case, the data processor 320 may change the location of the second reference line 912 such that the distance from the center line 914 to the first reference line 911 is identical to the distance from the center line 914 to the second reference line 912. The data processor 320 may generate a sagittal plane image 930 corresponding to changed reference lines. The sagittal plane image 930 may include a first sagittal plane image 931 and a second sagittal plane image 932. For example, the data processor 320 may generate a first sagittal plane image 931 corresponding to a changed location of the first reference line 911 on the axial plane image 910. The data processor 320 may also generate a second sagittal plane image 932 corresponding to a changed location of the second reference line 912 on the axial plane image 910.

The data processor 320 may minutely adjust the location of the second reference line 912 when changing the location of the second reference line 912 such that the distance from the center line 914 to the first reference line 911 is identical to the distance from the center line 914 to the second reference line 912. For example, the data processor 320 may generate the first sagittal plane image 931 corresponding to the changed location of the first reference line 911 on the axial plane image 910. The data processor 320 may also generate the second sagittal plane image 932 corresponding to the changed location of the second reference line 912 on the axial plane image 910. The data processor 320 may compare second sagittal plane images corresponding to a plurality of locations within a predetermined distance from the changed location of the second reference line 912 with the first sagittal plane image 931. The data processor 320 may select a second sagittal plane image that is the most similar to the first sagittal plane image 931, from the second sagittal plane images. The data processor 320 may also select a location of the second reference line 912 corresponding to the selected second sagittal plane image as a final location of the second reference line 912. The medical imaging apparatus 300 may display the final location of the second reference line 912 on the axial plane image 910.

FIGS. 10A and 10B illustrate a change in a sagittal plane image according to a movement of a reference line on an axial plane image according to an embodiment of the present disclosure.

A description of FIGS. 10A and 10B that is the same as given above with reference to FIGS. 9A and 9B will not be repeated herein.

Referring to FIG. 10A, the medical imaging apparatus 300 may display a center line 1014 on an axial plane image 1010 of an object 1015. The medical imaging apparatus 300 may also display a first reference line 1011 and a second reference line 1012 on the axial plane image 1010.

The input unit 340 may receive a user input for designating a location of a reference line on the axial plane image 1010. The data processor 320 may change the location of the first reference line 1011 or the location of the second reference line 1012, based on the user input.

Referring to FIG. 10B, the medical imaging apparatus 300 may display a sagittal plane image 1020. The data processor 320 may generate a first sagittal plane image 1021 corresponding to a location of the first reference line 1011 on the axial plane image 1010. The data processor 320 may also generate a second sagittal plane image 1022 corresponding to a location of the second reference line 1012 on the axial plane image 1010.

A user may change the location of the first reference line 1011 by using an indicator 1013. Since the location of the second reference line 1012 is not changed, a distance from the center line 1014 to the first reference line 1011 may become different from a distance from the center line 1014 to the second reference line 1012. The data processor 320 may generate a sagittal plane image 1030 corresponding to a changed first reference line 1011. The sagittal plane image 1030 may include a first sagittal plane image 1031 and a second sagittal plane image 1032. For example, the data processor 320 may generate a first sagittal plane image 1031 corresponding to a changed location of the first reference line 1011 on the axial plane image 1010. The data processor 320 may also generate a second sagittal plane image 1032 corresponding to the original location of the second reference line 1012 on the axial plane image 1010. As described above, a user may freely change the location of at least one reference line according to necessity. Thus, the user may more easily observe an image of the object 1015.

The data processor 320 may automatically adjust the location of the second reference line 1012. For example, the data processor 320 may generate the first sagittal plane image 1031 corresponding to the changed location of the first reference line 1011 on the axial plane image 1010. The data processor 320 may compare second sagittal plane images corresponding to a plurality of locations within a predetermined distance from the location of the second reference line 1012 with the first sagittal plane image 1031. The data processor 320 may select a second sagittal plane image that is the most similar to the first sagittal plane image 1031, from the second sagittal plane images. The data processor 320 may also select a location of the second reference line 1012 corresponding to the selected second sagittal plane image as a final location of the second reference line 1012. The medical imaging apparatus 300 may display the final location of the second reference line 1012 on the axial plane image 1010.

FIGS. 11A and 11B illustrate a change in an axial plane image according to a movement of a reference line on a sagittal plane image according to an embodiment of the present disclosure.

Referring to FIG. 11A, the medical imaging apparatus 300 may display a sagittal plane image 1110 of an object. Although only the sagittal plane image 1110 is displayed in FIG. 11A, the sagittal plane image 1110 may be displayed together with am axial plane image, a subtraction image, and a 3D image as in FIG. 8. The sagittal plane image 1110 may include a first sagittal plane image 1111 corresponding to a left breast of the object and a second sagittal plane image 1112 corresponding to a right breast of the object.

The medical imaging apparatus 300 may display a third reference line 1113 on the sagittal plane image 1110. The input unit 340 may receive a user input. The data processor 320 may move an indicator 1115, based on the user input. A user may change the location of the third reference line 1113 by using the indicator 1115.

The input unit 340 may receive a user input for designating a location of a reference line on the sagittal plane image 1110. The data processor 320 may change a location of the third reference line 1113, based on the user input.

For example, a user may change the location of the third reference line 1113 to the location of a changed third reference line 1114 by using the indicator 1115. In this case, an axial plane image 1120 may be changed to an axial plane image 1130.

Referring to FIG. 11B, the medical imaging apparatus 300 may display the axial plane image 1120. Although only the axial plane image 1120 is displayed in FIG. 11B, the axial plane image 1120 may be displayed together with the sagittal plane image 1110, a subtraction image, and a 3D image as in FIG. 8.

The data processor 320 may generate an axial plane image 1120 corresponding to a location of the third reference line 1113 on the sagittal plane image 1110. A user may change the location of the third reference line 1113 by using the indicator 1115. In this case, the data processor 320 may generate a changed axial plane image 1130 corresponding to the changed third reference line 1114. The axial plane image 1120 may display a section of an object 1121 corresponding to the third reference line 1113 on the sagittal plane image 1110. The changed axial plane image 1130 may display an axial section of an object 1131 corresponding to the changed third reference line 1114 on the sagittal plane image 1110.

FIGS. 12A and 12B illustrate a change in a subtraction image according to a movement of a reference line on a sagittal plane image according to an embodiment of the present disclosure.

As described above, the subtraction image is a type of axial plane image that more minutely represents tissues and/or blood vessels by using a contrast medium.

Referring to FIG. 12A, the medical imaging apparatus 300 may display a sagittal plane image 1210 of an object. Although only the sagittal plane image 1210 is displayed in FIG. 12A, the sagittal plane image 1210 may be displayed together with an axial plane image, a subtraction image, and a 3D image as in FIG. 8. The sagittal plane image 1210 may include a first sagittal plane image 1211 corresponding to a left breast of the object and a second sagittal plane image 1212 corresponding to a right breast of the object.

The medical imaging apparatus 300 may display a fourth reference line 1213 on the sagittal plane image 1210. Although only the fourth reference line 1213 is illustrated in FIG. 12A, the medical imaging apparatus 300 may display the third reference line 1113 of FIG. 11A and the fourth reference line 1213 at the same time. The third reference line 1113 and the fourth reference line 1213 may be the same reference line. Accordingly, the data processor 320 may generate both a sagittal plane image and a subtraction image in correspondence to a reference line on the sagittal plane image 1210.

The input unit 340 may receive a user input. The data processor 320 may move an indicator 1215, based on the user input. A user may change the location of the fourth reference line 1213 by using the indicator 1215.

The input unit 340 may receive a user input for designating a location of a reference line on the sagittal plane image 1210. The data processor 320 may change a location of the fourth reference line 1213, based on the user input.

For example, a user may change the location of the fourth reference line 1213 to the location of a changed fourth reference line 1214 by using the indicator 1215. The medical imaging apparatus 300 may not display the fourth reference line 1213 but may display only the changed fourth reference line 1214. Due to the change in the fourth reference line, the subtraction image 1220 may be changed to a subtraction image 1230.

Referring to FIG. 12B, the medical imaging apparatus 300 may display the subtraction image 1220. Although only the subtraction image 1220 is displayed in FIG. 12B, the subtraction image 1220 may be displayed together with the sagittal plane image 1210, an axial plane image, and a 3D image as in FIG. 8.

The data processor 320 may generate a subtraction image 1220 corresponding to a location of the fourth reference line 1213 on the sagittal plane image 1210. A user may change the location of the fourth reference line 1213 by using the indicator 1215. In this case, the data processor 320 may generate a changed axial plane image 1230 corresponding to the changed fourth reference line 1214. The subtraction image 1220 may display a section of an object 1221 corresponding to the fourth reference line 1213 on the sagittal plane image 1210. The changed subtraction image 1230 may display an axial section of an object 1231 corresponding to the changed fourth reference line 1214 on the sagittal plane image 1210.

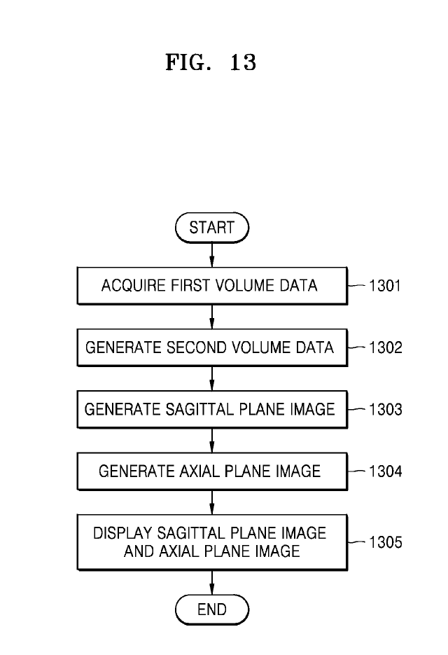

FIG. 13 is a flowchart of a method of operating a medical imaging apparatus according to an embodiment of the present disclosure.

Operation 1301 may be performed in the data acquirer 310. Operations 1302, 1302, and 1304 may be performed in the data processor 320. Operation 1305 may be performed in the display 330.

In operation 1301, the medical imaging apparatus 300 acquires first volume data including first sections of a first part and a second part of an object. In operation 1302, the medical imaging apparatus 300 generates second volume data including second sections of the first part and the second part of the object, based on the first volume data. In operation 1303, the medical imaging apparatus 300 generates a sagittal plane image of the first part and the second part of the object, based on the first volume data or the second volume data. In operation 1304, the medical imaging apparatus 300 generates an axial plane image of a third part including the first part and the second part, based on the first volume data or the second volume data. In operation 1305, the medical imaging apparatus 300 displays a UI screen image including the sagittal plane image and the axial plane image.

Operation 1305 includes displaying at least one reference line on each of the sagittal plane image and the axial plane image. Operation 1304 may include generating an axial plane image corresponding to a location of a reference line on the sagittal plane image.

The medical imaging apparatus 300 may perform an operation of generating a subtraction image corresponding to a difference between an axial plane image before injecting a contrast medium and an axial plane image after injecting a contrast medium. The medical imaging apparatus 300 may perform an operation of displaying the subtraction image. The subtraction image may be an image corresponding to a location of a reference line on the sagittal plane image.

The sagittal plane image may include a first sagittal plane image corresponding to the first part of the object and a second sagittal plane image corresponding to the second part of the object. Operation 1303 may include generating a first sagittal plane image corresponding to a location of a first reference line on the axial plane image. Operation 1303 may include generating a second sagittal plane image corresponding to a location of a second reference line on the axial plane image.

The medical imaging apparatus 300 may perform an operation of setting a distance from a center line on the axial plane image to the first reference line to be identical to a distance from the center line on the axial plane image to a second reference line.

The first part may be a left breast of the object, and the second part may be a right breast thereof. The medical imaging apparatus 300 may perform an operation of arranging a chest wall of the object on the first sagittal plane image and a chest wall of the object on the second sagittal plane image such that the two chest walls face each other.

The medical imaging apparatus 300 may perform an operation of receiving a user input for designating a location of a reference line. The medical imaging apparatus 300 may perform an operation of changing the location of the reference line according to the user input. The operation of receiving the user input may include an operation of receiving information about a location of the center line on the axial plane image.

When the first sections are sagittal sections, the second sections may be axial sections. When the first sections are axial sections, the second sections may be sagittal sections.

The medical imaging apparatus 300 may perform an operation of generating sagittal plane images of a plurality of left breasts and sagittal plane images of a plurality of right breasts, based on the first volume data or the second volume data.

The medical imaging apparatus 300 may perform an operation of selecting a sagittal plane image of a right breast that is the most similar to one of the sagittal plane images of the plurality of left breasts. The medical imaging apparatus 300 may perform an operation of displaying the selected sagittal plane image of the right breast and the sagittal plane image of the left breast that is the most similar to the selected sagittal plane image.

FIGS. 14A and 14B illustrate screen images that may be displayed on the display 330 of the medical imaging apparatus 300 according to an embodiment of the present disclosure.

Referring to FIG. 14A, the display 330 may display a sagittal plane image 1410 including a first sagittal plane image 1411 and a second sagittal plane image 1412. The display 330 may display a chest wall 1413 on the first sagittal plane image 1411. The display 330 may display a chest wall 1414 on the second sagittal plane image 1412. The first sagittal plane image 1411 may be an image of a left breast. The second sagittal plane image 1412 may be an image of a right breast.

The medical imaging apparatus 300 may acquire a plurality of volume data acquired at different times. The medical imaging apparatus 300 may display the sagittal plane image 1410, based on one of the plurality of volume data. The display 330 may display a time line 1420. A user may move a button 1421 on the screen image by using the input unit 340. The medical imaging apparatus 300 may display a sagittal plane image 1410 corresponding to a location of the button 1421. The medical imaging apparatus 300 may change at least one of the first and second sagittal plane images 1411 and 1412 corresponding to the location of the button 1421 to a sagittal plane image acquired at a different time. A user may observe a change in the sagittal plane image 1410 over time.

The display 330 may display the change in the at least one of the first and second sagittal plane images 1411 and 1412 over time. The display 330 may display a selection list 1422. A user may select a left breast, a right breast, or left and right breasts from the selection list 1422 by using the input unit 340. For example, when the left breast is selected, the medical imaging apparatus 300 may display a change in the first sagittal plane image 1411 over time according to a location of the button 1421.

Referring to FIG. 14B, the display 330 may display a sagittal plane image 1430. A user may move a button 1441 on a time line 1440 by using the input unit 340. The medical imaging apparatus 300 may display a first sagittal plane image 1431 having a first chest wall 1433 and a second sagittal plane image 1432 having a second chest wall 1434 corresponding to a location of the button 1441. A user may select a left breast, a right breast, or left and right breasts from a selection list 1442 by using the input unit 340. For example, when the left breast is selected, the medical imaging apparatus 300 may display a change in the first sagittal plane image 1431 over time according to a location of the button 1441.

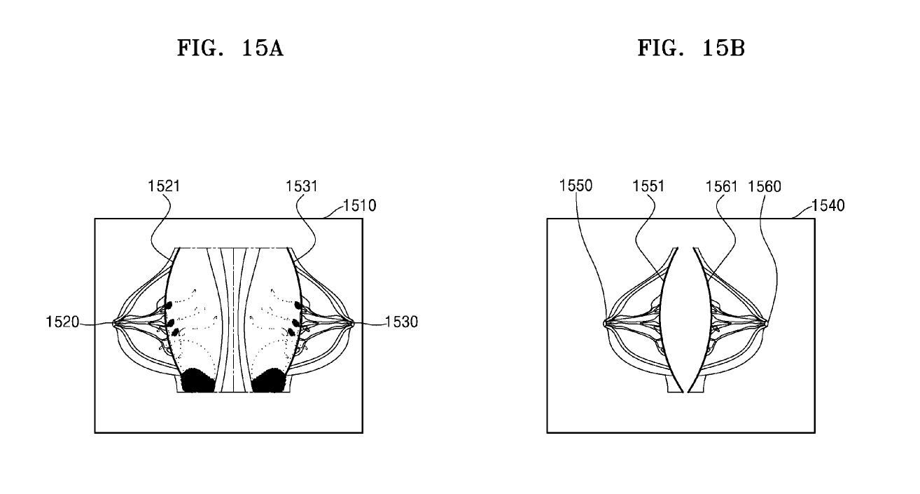

FIGS. 15A and 15B illustrate sagittal plane images according to an embodiment of the present disclosure.

Referring to FIG. 15A, the display 330 may display a sagittal plane image 1510. The sagittal plane image 1510 may display a sagittal plane image 1520 of a left breast and a sagittal plane image 1530 of a right breast. The medical imaging apparatus 300 may also display chest walls 1521 and 1531 on the display 330 by performing image processing. Alternatively, the medical imaging apparatus 300 may display the chest walls 1521 and 1531 on the display 330, based on a user input.

Referring to FIG. 15B, the display 330 may display a sagittal plane image 1550 of the left breast and a sagittal plane image 1560 of the right breast. The display 330 may display chest walls 1551 and 1561. The medical imaging apparatus 300 may adjust a distance between the chest wall 1551 of the left breast and the chest wall 1561 of the right breast. For example, the medical imaging apparatus 300 may set the distance between the chest wall 1551 of the left breast and the chest wall 1561 of the right breast, based on a signal of the input unit 340 manipulated by a user. The user may adjust the distance between the chest wall 1551 of the left breast and the chest wall 1561 of the right breast by dragging the sagittal plane images 1550 and 1560 by using a mouse. The user may input the distance between the chest walls 1551 and 1561. The medical imaging apparatus 300 may set a predetermined distance regardless of a user input.

Since the sagittal plane image 1550 of the left breast and the sagittal plane image 1560 of the right breast included in a sagittal plane image 1540 show both a heart area and a lung area, a user may be hindered in observing a lesion in the breasts. The medical imaging apparatus 300 reduces the distance between the chest wall 1551 of the left breast and the chest wall 1561 of the right breast so that a user may easily compare the sagittal plane image 1550 of the left breast with the sagittal plane image 1560 of the right breast.

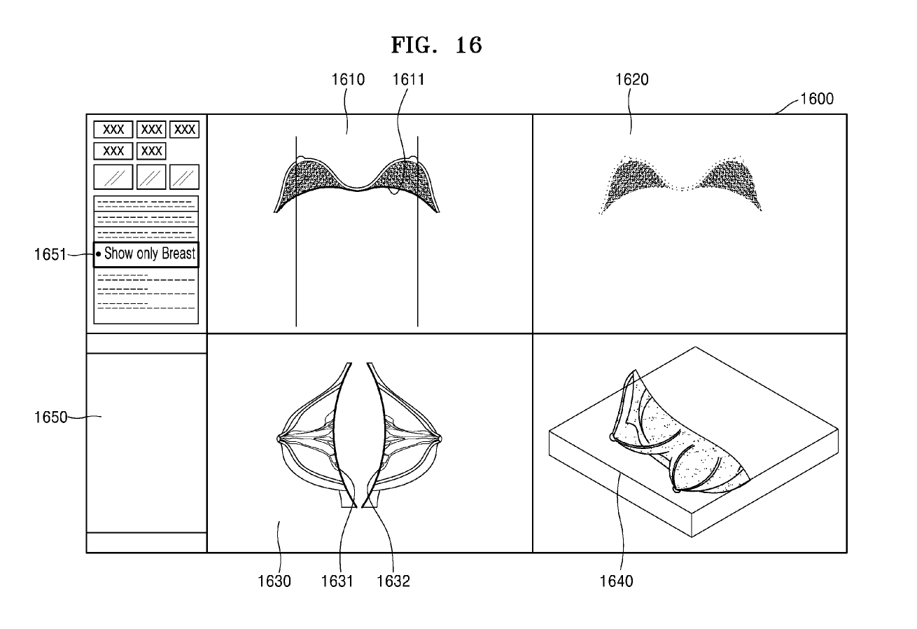

FIG. 16 illustrates a screen image that may be displayed on the display 330 of the medical imaging apparatus 300 according to an embodiment of the present disclosure.

Referring to FIG. 16, the medical imaging apparatus 300 may display a UI screen image 1600 including an axial plane image 1610, a subtraction image 1620, a sagittal plane image 1630, a 3D image 1640, and a menu 1650.

A user may variously set a medical image by using the menu 1650. The user may set the medical image by using the menu 1650 such that only breasts are displayed. For example, the user may click a ‘show only breast’ button 1651 by using the input unit 340. The medical imaging apparatus 300 may tick a box beside the ‘show only breast’ button 1651. The medical imaging apparatus 300 may determine an area of a chest wall by performing image processing with respect to at least one the first volume data and the second volume data. The medical image may be divided into a breast area and a lung area, based on a chest wall 1611.

The medical imaging apparatus 300 may display only a breast side of the chest wall 1611 on the display 330. For example, the medical imaging apparatus 300 may display an axial plane image 1610 of the breast side of the chest wall 1611. Compared with FIG. 8, since the heart area and the lung area are not shown on the axial plane image 1610, a user may easily determine a lesion of a subject.

The medical imaging apparatus 300 may display the subtraction image of the breast side of the chest wall 1611. The medical imaging apparatus 300 may display the sagittal plane image 1630 of breast sides of chest walls 1631 and 1632. The medical imaging apparatus 300 may display the 3D image 1640 of a breast side of a chest wall.

A user may click again the ‘show only breast’ button 1651 by using the input unit 340. The medical imaging apparatus 300 may remove a tick from the box beside the ‘show only breast’ button 1651. The medical imaging apparatus 300 may display FIG. 8 on the display 330 by removing the tick from the box beside the ‘show only breast’ button 1651.

According to a medical imaging apparatus, a method of operating the medical imaging apparatus, and a recording medium having the operating method of the medical imaging apparatus recorded thereon as described above, a user may simultaneously check images of a plurality of parts of an object. The user may make a more easy diagnose by comparing the images of the plurality of parts. For example, in the case of a body part that is divided into a left side and a right side, such as breasts, both side breasts may be compared with each other on one screen image. Since a user is able to more easily search for a desired part via a movement of a reference line, the user may more easily diagnose a bilateral lesion.

The embodiments of the present disclosure can be written as computer programs and can be implemented in general-use digital computers that execute the programs using a non-transitory computer readable recording medium. Examples of the non-transitory computer readable recording medium include magnetic storage media (e.g., read-only memory (ROM), floppy disks, hard disks, etc.), optical recording media (e.g., CD-ROMs, or DVDs), etc.

At least a portion of a “˜unit” may be implemented as hardware. The hardware may include a processor. The processor may be a general-purpose single- or multi-chip microprocessor (for example, an Advanced RISC Machine (ARM)), a special-purpose microprocessor (for example, a digital signal processor (DSP)), a micro-controller, a programmable gate array, or the like. The processor may be referred to as a central processing unit (CPU). At least a portion of a “˜unit” may be a combination (for example, an ARM and a DSP) of processors.

The hardware may also include a memory. The memory may be an arbitrary electronic component capable of storing electronic information. The memory may be implemented as a random access memory (RAM), a ROM, a magnetic disk storage medium, an optical storage medium, a flash memory device within the RAM, an on-board memory included in a processor, an erasable programmable ROM (EPROM), an Electrically Erasable Programmable ROM (EEPROM), registers, or combinations thereof.

Data and commands may be stored in the memory. The commands are executable by a processor to implement the methods disclosed in the present application. Execution of the commands may include use of the data stored in the memory. When the processor executes the commands, various parts of the commands may be loaded onto the processor, and various pieces of the data may be loaded onto the processor.

The exemplary embodiments should be considered in descriptive sense only and not for purposes of limitation. Descriptions of features or aspects within each embodiment should typically be considered as available for other similar features or aspects in other embodiments.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A medical imaging apparatus comprising:
a data acquirer configured to acquire first volume data including first sections of a left breast and a right breast of an object;
an image processor configured to:
generate second volume data including second sections of the left breast and the right breast of the object, based on the first volume data;
generate a sagittal plane image of the left breast and the right breast, based on the first volume data or the second volume data; and
generate an axial plane image of beasts including the left breast and the right breast, based on the first volume data or the second volume data; and
a display configured to display a user interface (UI) screen image including the sagittal plane image and the axial plane image.

2. The medical imaging apparatus of claim 1, wherein the display is further configured to display at least one reference line on each of the sagittal plane image and the axial plane image.

3. The medical imaging apparatus of claim 2, wherein the image processor is further configured to generate the axial plane image corresponding to a location of the at least one reference line on the sagittal plane image.

4. The medical imaging apparatus of claim 2, wherein
the image processor is further configured to generate a subtraction image corresponding to a difference between an axial plane image before injecting a contrast medium and an axial plane image after injecting a contrast medium,
the subtraction image is an image corresponding to a location of the at least one reference line on the sagittal plane image, and
the display is further configured to display the subtraction image.

5. The medical imaging apparatus of claim 2, wherein
the sagittal plane image comprises a first sagittal plane image corresponding to the left breast of the object and a second sagittal plane image corresponding to the right breast of the object, and
the image processor is further configured to generate the first sagittal plane image corresponding to a location of a first reference line on the axial plane image, and generate the second sagittal plane image corresponding to a location of a second reference line on the axial plane image.

6. The medical imaging apparatus of claim 5, wherein the image processor is further configured to set a distance from a center line on the axial plane image to the first reference line to be identical to a distance from the center line on the axial plane image to the second reference line.

7. The medical imaging apparatus of claim 5, wherein the image processor is further configured to arrange a first chest wall of the object on the first sagittal plane image and a second chest wall of the object on the second sagittal plane image such that the first chest wall and the second chest wall face each other.

8. The medical imaging apparatus of claim 2, further comprising an input unit configured to receive a user input for designating a location of the at least one reference line,
wherein the image processor is further configured to change the location of the at least one reference line based on the user input, and
wherein the input unit is further configured to receive information about a location of a center line on the axial plane image.

9. The medical imaging apparatus of claim 1, wherein
when the first sections are sagittal sections, the second sections are axial sections; and
when the first sections are axial sections, the second sections are sagittal sections.

10. The medical imaging apparatus of claim 1, wherein
the image processor is further configured to:
generate a plurality of sagittal plane images of the left breast and a plurality of sagittal plane images of the right breast, based on the first volume data or the second volume data; and
select, from among the plurality of sagittal plane images of the right breast, a sagittal plane image of the right breast that is most similar to a sagittal plane image of the left breast, and
the display is further configured to display the selected sagittal plane image of the right breast and the sagittal plane image of the left breast.

11. A method of operating a medical imaging apparatus, the method comprising:
acquiring first volume data including first sections of a left breast and a right breast of an object;
generating second volume data including second sections of the left breast and the right breast of the object, based on the first volume data;
generating a sagittal plane image of the left breast and the right breast, based on the first volume data or the second volume data;
generating an axial plane image of breasts including the left breast and the right breast, based on the first volume data or the second volume data; and
displaying a user interface (UI) screen image including the sagittal plane image and the axial plane image.

12. The method of claim 11, wherein the displaying of the UI screen image comprises displaying at least one reference line on each of the sagittal plane image and the axial plane image.

13. The method of claim 12, wherein the generating of the axial plane image comprises generating the axial plane image corresponding to a location of the at least one reference line on the sagittal plane image.

14. The method of claim 12, further comprising:
generating a subtraction image corresponding to a difference between an axial plane image before injecting a contrast medium and an axial plane image after injecting a contrast medium; and
displaying the subtraction image,
wherein the subtraction image is an image corresponding to a location of the at least one reference line on the sagittal plane image.

15. The method of claim 12, wherein
the sagittal plane image comprises a first sagittal plane image corresponding to the left breast of the object and a second sagittal plane image corresponding to the right breast of the object, and
the generating of the sagittal plane image comprises:
generating the first sagittal plane image corresponding to a location of a first reference line on the axial plane image; and
generating the second sagittal plane image corresponding to a location of a second reference line on the axial plane image.

16. The method of claim 15, further comprising setting a distance from a center line on the axial plane image to the first reference line to be identical to a distance from the center line on the axial plane image to the second reference line.

17. The method of claim 15, further comprising arranging a first chest wall of the object on the first sagittal plane image and a second chest wall of the object on the second sagittal plane image such that the first chest wall and the second chest wall face each other.

18. The method of claim 12, further comprising:
receiving a user input for designating a location of the at least one reference line; and
changing the location of the at least one reference line based on the user input, and
wherein the receiving of the user input comprises receiving information about a location of a center line on the axial plane image.

19. The method of claim 11, wherein
the generating of the sagittal plane image comprises generating a plurality of sagittal plane images of the left breast and a plurality of sagittal plane images of the right breast, based on the first volume data or the second volume data, and
the displaying comprises:
selecting, from among the plurality of sagittal plane images of the right breast, a sagittal plane image of the right breast that is most similar to a sagittal plane image of the left breast, and
displaying the selected sagittal plane image of the right breast and the sagittal plane image of the left breast.
